(19) United States
(12) Patent Application Publication
(10) Pub. No.: US 2025/0032096 A1
(43) Pub. Date: Jan. 30, 2025

(54) ULTRASOUND DIAGNOSTIC APPARATUS AND PROGRAM

(71) Applicant: FUJIFILM Healthcare Corporation, Chiba (JP)

(72) Inventor: NATSUMI DANNO, Chiba (JP)

(73) Assignee: FUJIFILM Healthcare Corporation, Chiba (JP)

(21) Appl. No.: 18/764,377

(22) Filed: Jul. 5, 2024

(30) Foreign Application Priority Data
Jul. 25, 2023 (JP) 2023-120891

(51) Int. Cl. A61B 8/00

(52) U.S. Cl. CPC A61B 8/565; A61B 8/463; A61B 8/54

(57) ABSTRACT

An ultrasound diagnostic apparatus including an acquisition unit and a linking unit is provided. An ultrasonic examination is executed by the acquisition unit to acquire ultrasound data. The linking unit links first ultrasound data, which is acquired by executing an ultrasonic examination in accordance with a pre-registered examination procedure, with an examination process corresponding to the ultrasonic examination. The linking unit further links second ultrasound data, which is acquired by executing an ultrasonic examination in a procedure different from the pre-registered examination procedure, with an examination process corresponding to the ultrasonic examination.

### CROSS-REFERENCE TO RELATED APPLICATION

This application claims the priority benefit of Japan application serial no. 2023-120891, filed on Jul. 25, 2023. The entirety of the above-mentioned patent application is hereby incorporated by reference herein and made a part of this specification.

### BACKGROUND

### 1. Technical Field

The present disclosure relates to an ultrasound diagnostic apparatus and a program.

### 2. Description of the Related Art

In general, an examination using an ultrasound diagnostic apparatus includes a plurality of examination processes. For example, a procedure of the plurality of examination processes is pre-registered in the ultrasound diagnostic apparatus, and an examination (for example, capturing of a B-mode image or Doppler imaging) corresponding to each examination process is performed in accordance with the procedure.

JP2015-173667A discloses a device that extracts a portion to be corrected for an arrangement order of a plurality of image data.

JP2006-280640A discloses a device that superimposes and displays examination identification data on each examination mode image data.

### SUMMARY OF THE INVENTION

In a case where an ultrasonic examination is executed in accordance with a pre-registered examination procedure, ultrasound data acquired by executing the ultrasonic examination may be linked to an examination process corresponding to the ultrasonic examination.

In the related art, in a case where the ultrasonic examination is executed in a procedure different from the pre-registered examination procedure, ultrasound data acquired by executing the ultrasonic examination is simply stored in accordance with an order of the acquisition. For example, after a certain examination is executed, additional ultrasound data may be acquired by executing an additional examination. In this case, the additional ultrasound data is stored in accordance with an order of the acquisition. Therefore, it may be difficult for a user (for example, a doctor or an examination technician) to understand which examination process the ultrasound data belongs to.

An object of the present disclosure is to organize and manage ultrasound data acquired by executing an ultrasonic examination in a procedure different from a pre-registered examination procedure.

According to one aspect of the present disclosure, there is provided an ultrasound diagnostic apparatus comprising: an acquisition unit that acquires ultrasound data by executing an ultrasonic examination; and a linking unit that links first ultrasound data, which is acquired by the acquisition unit by executing the ultrasonic examination in accordance with a pre-registered examination procedure, with an examination process corresponding to the ultrasonic examination, in which the linking unit further links second ultrasound data, which is acquired by executing the ultrasonic examination in a procedure different from the pre-registered examination procedure, with an examination process corresponding to the ultrasonic examination.

The linking unit may link an examination process designated by a user with the second ultrasound data.

The ultrasound diagnostic apparatus may further include a display controller that displays, on a display unit, an image schematically showing an examination site that is a candidate for the ultrasonic examination. The linking unit may link an examination process corresponding to the examination site designated by the user from the image with the second ultrasound data.

The ultrasound diagnostic apparatus may further include a display controller, and the display controller may arrange and display the first ultrasound data on a display unit in accordance with an order of the examination processes linked to the first ultrasound data, and arrange and display the second ultrasound data on the display unit in accordance with an order of the examination processes linked to the second ultrasound data. In a case where the user designates an examination process on a screen on which the first ultrasound data and the second ultrasound data are displayed, the linking unit may correct the examination process linked to the second ultrasound data by linking the examination process designated by the user on the screen with the second ultrasound data.

In a case where the second ultrasound data is acquired by the acquisition unit, information indicating the examination process in which the second ultrasound data is acquired may be attached to the second ultrasound data as accessory information. In a case where the examination process indicated by the accessory information is different from the examination process designated by the user, the linking unit may correct the examination process linked to the second ultrasound data by linking the examination process indicated by the accessory information with the second ultrasound data.

The ultrasound diagnostic apparatus may further include a display controller, and the display controller may arrange and display, on a display unit, an ultrasound data group before the examination process is corrected and an ultrasound data group after the examination process is corrected. The ultrasound data groups may include the first ultrasound data and the second ultrasound data.

The ultrasound diagnostic apparatus may further include an analysis unit that analyzes the second ultrasound data to specify the examination process corresponding to the ultrasonic examination in which the second ultrasound data is obtained. The linking unit may link the examination process specified by the analysis unit with the second ultrasound data.

The ultrasound diagnostic apparatus may further include a display controller, and the display controller may display the first ultrasound data on a display unit in accordance with an order of examination processes, display the second ultrasound data on the display unit in accordance with an order of examination processes designated by a user, and display the second ultrasound data on the display unit in accordance with an order of the examination processes which are specified by the analysis unit and correspond to the ultrasonic examination. In a case where the user designates an examination process on a screen on which the first ultrasound data and the second ultrasound data are displayed, the linking unit may correct the examination process linked to the second ultrasound data by linking the examination process designated by the user on the screen with the second ultrasound data.

According to one aspect of the present disclosure, there is provided a program for causing a computer to function as: an acquisition unit that acquires ultrasound data by executing an ultrasonic examination; and a linking unit that links first ultrasound data, which is acquired by the acquisition unit by executing the ultrasonic examination in accordance with a pre-registered examination procedure, with an examination process corresponding to the ultrasonic examination, in which the linking unit further links second ultrasound data, which is acquired by executing the ultrasonic examination in a procedure different from the pre-registered examination procedure, with an examination process corresponding to the ultrasonic examination.

According to the present disclosure, it is possible to organize and manage ultrasound data acquired by executing an ultrasonic examination in a procedure different from a pre-registered examination procedure.

### BRIEF DESCRIPTION OF THE DRAWINGS

FIG. 1 is a block diagram showing a configuration of an ultrasound diagnostic apparatus according to an embodiment.

FIG. 2 is a diagram showing a screen on which an ultrasound image and a view list are displayed.

FIG. 3 is a diagram for describing Example 1.

FIG. 4 is a diagram for describing Example 1.

FIG. 5 is a diagram showing a view list.

FIG. 6 is a diagram showing a display example of an ultrasound image.

FIG. 7 is a diagram showing a display example of a view list and an ultrasound image.

### DESCRIPTION OF THE EMBODIMENTS

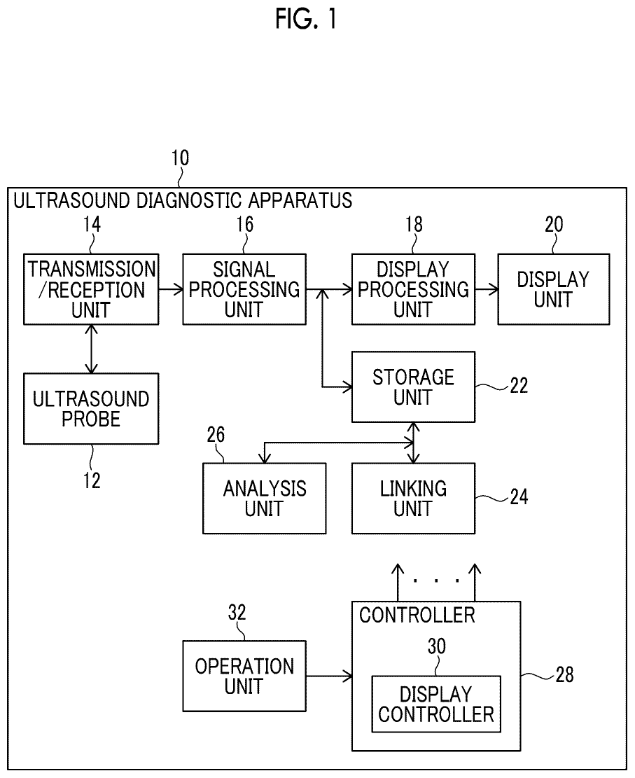

An ultrasound diagnostic apparatus 10 according to an embodiment will be described with reference to FIG. 1. FIG. 1 shows an example of a configuration of the ultrasound diagnostic apparatus 10 according to the embodiment.

The ultrasound diagnostic apparatus 10 generates an ultrasound image representing an internal tissue of a subject by transmitting ultrasonic waves into a subject through an ultrasound probe 12 and receiving the ultrasonic waves reflected in the subject.

The ultrasound probe 12 is a device that transmits and receives the ultrasonic waves. The ultrasound probe 12 includes, for example, a 1D array oscillator. The 1D array oscillator includes a plurality of ultrasound oscillators arranged in one dimension. An ultrasonic beam is formed by the 1D array oscillator, and electron scanning with the ultrasonic beam is repeatedly performed. As a result, a scanning plane is formed in a living body for each electron scanning. The ultrasound probe 12 may include, instead of the 1D array oscillator, a 2D array oscillator formed to include a plurality of ultrasound oscillators arranged in two dimensions. As a scanning method, sector scanning, linear scanning, convex scanning, or the like is used. For example, the ultrasound probe 12 includes an ultrasound oscillator.

A transmission/reception unit 14 functions as a transmission beam former and a reception beam former. In the transmission, the transmission/reception unit 14 supplies a plurality of transmission signals having a certain delay relationship to the plurality of ultrasound oscillators included in the ultrasound probe 12. As a result, a transmission beam of the ultrasonic wave is formed. In the reception, a reflected wave (RF signal) from the living body is received by the ultrasound probe 12, whereby a plurality of reception signals are output from the ultrasound probe 12 to the transmission/reception unit 14. The transmission/reception unit 14 forms a reception beam by applying phasing addition processing to the plurality of reception signals. The beam data is output to a signal processing unit 16. That is, the transmission/reception unit 14 forms the reception beam by performing delay processing on the reception signal obtained from each ultrasound oscillator in accordance with a delay processing condition for each ultrasound oscillator and performing addition processing on the plurality of reception signals obtained from the plurality of ultrasound oscillators. The delay processing condition is defined by reception delay data indicating a delay time. A reception delay data set (that is, a set of delay times) corresponding to the plurality of ultrasound oscillators is supplied from a controller 28.

The signal processing unit 16 generates an ultrasound image (for example, a B-mode image) by applying, to the beam data output from the transmission/reception unit 14, signal processing such as detection, amplitude compression (amplitude transform) such as logarithmic compression, and a conversion function (a coordinate transformation function and an interpolation processing function by a digital scan converter (DSC)). The ultrasound image is not limited to the B-mode image, and may be a color Doppler image, a pulse Doppler image, a strain image, a shear wave elastography image, or the like.

The transmission/reception unit 14 and the signal processing unit 16 correspond to an example of an acquisition unit.

A display processing unit 18 generates a display image by overlaying necessary graphic data on the ultrasound image. The display image is output to a display unit 20. One or a plurality of images are arranged and displayed in a display aspect according to a display mode.

The display unit 20 is a display such as a liquid crystal display or an EL display. The ultrasound image such as the B-mode image is displayed on the display unit 20. The display unit 20 may be a device comprising both the display and an operation unit 32. For example, a graphic user interface (GUI) may be realized by the display unit 20. In addition, a user interface such as a touch panel may be realized by the display unit 20.

A storage unit 22 constitutes one or a plurality of storage regions for storing data. The storage unit 22 is, for example, a hard disk drive (HDD), a solid state drive (SSD), various memories (for example, RAM, DRAM, or ROM), other storage devices (for example, optical disk), or a combination thereof. The image generated through the imaging of the ultrasound diagnostic apparatus 10, information indicating an imaging condition, information regarding a patient, and the like are stored in the storage unit 22.

A linking unit 24 links ultrasound data, which is acquired by executing an ultrasonic examination in accordance with a pre-registered examination procedure, with an examination process corresponding to the ultrasonic examination. For example, the linking unit 24 links ultrasound data with information indicating the examination process corresponding to the ultrasonic examination, and stores the ultrasound data and the information in the storage unit 22.

The ultrasound data is the beam data output from the transmission/reception unit 14, the data (that is, the ultrasound image) output from the signal processing unit 16, or the data (that is, the display image) output from the display processing unit 18.

The ultrasonic examination is an examination for acquiring the ultrasound data, and is, for example, B-mode image capturing or Doppler imaging. Of course, imaging and data acquisition other than the B-mode image capturing or Doppler imaging may be included in the ultrasonic examination.

An examination procedure is an order of various examinations for diagnosing the subject. For example, various examinations include an ultrasonic examination and other processing. The other processing includes displaying the ultrasound image, detecting an abnormality represented in the ultrasound image, displaying the abnormality, measuring the abnormality, adjusting the image, displaying or creating a report, and the like. Of course, various examinations may not include all or a part of the other processing.

The examination procedure is predetermined as an examination protocol. Information indicating the examination protocol (that is, information indicating the examination procedure) is previously stored in the storage unit 22. The examination protocol is created according to an examination purpose, an examination site, a medical department, or the like, and is configured of a combination of one or more examination processes. The examination process is a process corresponding to an ultrasonic examination or an examination other than the ultrasonic examination. The ultrasound diagnostic apparatus 10 operates in accordance with the examination protocol, thereby supporting work (for example, examination work or diagnostic work) of a user such as a doctor or an examination technician. The ultrasound diagnostic apparatus 10 has a protocol assistant function as an examination support function.

For example, the examination protocol is determined for each examination site or for each medical department. Information indicating the examination protocol for each examination site or information indicating the examination protocol for each medical department is previously stored in the storage unit 22. The examination site is an abdomen, a chest, a heart, a blood vessel, a neck, a urinary organ, or the like. The medical department is an internal medicine department, a surgery department, an obstetrics and gynecology department, a radiology department, or the like. Of course, the examination site and the medical department are not limited to these.

In the examination protocol, an order in which each examination is to be executed is determined. For example, in the examination protocol, the fact that capturing the ultrasound image, detecting an abnormality represented in the ultrasound image, measuring the abnormality, and creating a report are executed in that order is determined. With regard to the capturing of the ultrasound image, a site, a cross section, a region, an imaging order, and the like to be captured by the ultrasonic waves are predetermined and are included in the examination protocol.

The analysis unit 26 analyzes the ultrasound data to specify the examination process corresponding to the ultrasonic examination in which the ultrasound data is obtained. For example, a cross section, a site, or the like to be subjected to the ultrasonic examination in the examination process is predetermined. The analysis unit 26 specifies a cross section or a site from which the ultrasound image is acquired by applying image analysis to the ultrasound image. As a technique of specifying a cross section or an examination site, artificial intelligence (AI) or pattern matching is used. For example, the analysis unit 26 specifies a cross section from which the ultrasound image is acquired by performing matching processing between the ultrasound image acquired by the ultrasonic examination and a typical ultrasound image of each cross section, for each cross section. The analysis unit 26 specifies an examination process in which the ultrasonic examination is executed for the specified cross section. The artificial intelligence may be generated through learning using the ultrasound image acquired by the ultrasonic examination, the cross section where the ultrasonic examination is performed, the site where the ultrasonic examination is performed, and the like. The analysis unit 26 specifies the cross section or the site from which the ultrasound data is acquired by using the artificial intelligence, and specifies an examination process in which the ultrasonic examination is executed for the specified cross section or site.

The controller 28 controls an operation of each unit of the ultrasound diagnostic apparatus 10.

The controller 28 includes a display controller 30. The display controller 30 displays the ultrasound data on the display unit 20. The display controller 30 may display information or an image other than the ultrasound data on the display unit 20.

The operation unit 32 is a device for the user to input a condition, a command, and the like necessary for imaging, to the ultrasound diagnostic apparatus 10. For example, the operation unit 32 is an operation panel, a switch, a button, a keyboard, a mouse, a track ball, or a joystick.

Hereinafter, the present embodiment will be described in detail.

The ultrasonic examination may be executed in a procedure different from a pre-registered examination procedure. That is, the ultrasonic examination that is not pre-registered in the examination protocol may be executed. For example, after the pre-registered examination procedure is completed, an additional ultrasonic examination may be executed in response to an instruction from the user such as the doctor or the examination technician. In addition, the ultrasonic examination that is not pre-registered in the examination protocol may be executed in the middle of the pre-registered examination procedure. In such a case, the linking unit 24 links ultrasound data acquired by executing the ultrasonic examination with an examination process corresponding to the ultrasonic examination. For example, the linking unit 24 links the ultrasound data with information indicating the examination process corresponding to the ultrasonic examination, and stores the ultrasound data and the information in the storage unit 22.

Hereinafter, for convenience of description, the ultrasound data acquired by executing the ultrasonic examination in accordance with the pre-registered examination procedure is referred to as “first ultrasound data”. That is, the ultrasound data acquired by executing the ultrasonic examination in accordance with the examination procedure that is pre-registered in the examination protocol is referred to as “first ultrasound data”. The ultrasound data acquired by executing the ultrasonic examination in a procedure different from the pre-registered examination procedure is referred to as “second ultrasound data”. That is, the ultrasound data acquired by executing the ultrasonic examination in accordance with the examination procedure that is not pre-registered in the examination protocol is referred to as “second ultrasound data”.

The examination process to which the second ultrasound data is linked may be an examination process according to the pre-registered examination procedure, or may be an additional examination process not included in the examination procedure. The linking unit 24 may link the second ultrasound data with an examination process designated by the user, or may link the second ultrasound data with an automatically selected examination process.

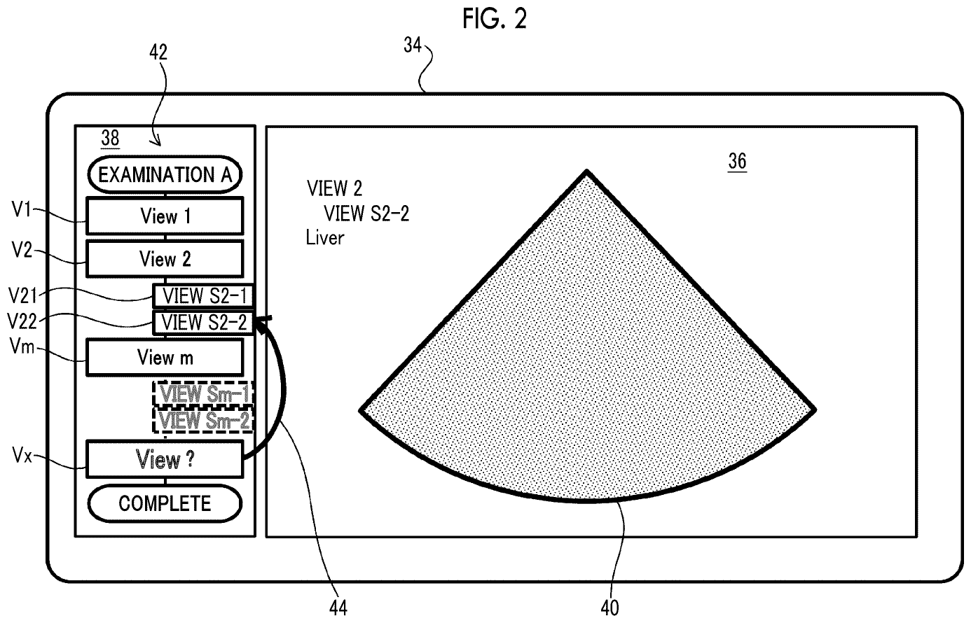

An example of the operation of linking the ultrasound data with the examination process will be described with reference to FIG. 2. FIG. 2 is a diagram showing an example of a screen on which an ultrasound image and a view list are displayed.

The display controller 30 displays an examination screen 34 on the display unit 20. The examination screen 34 includes a display region 36 and a display region 38. For example, the display controller 30 displays, on the display unit 20, the examination screen 34 in the middle of executing the ultrasonic examination (for example, in the middle of imaging by ultrasonic waves), after the ultrasonic examination is executed, or after the examination protocol is completed.

The display region 36 is a region in which the ultrasound data is displayed. In a case where the ultrasound data is acquired through the transmission and reception of the ultrasonic waves, the display controller 30 displays the ultrasound data in the display region 36. Here, as an example, a B-mode image 40 is displayed in the display region 36 as an example of the ultrasound data.

The display region 38 is a region in which a view list 42 is displayed. The view list 42 includes one or a plurality of views. The view is an examination process. The view list 42 is a list representing examination processes included in the examination protocol. One or a plurality of views are arranged in the view list 42 in accordance with the examination procedure.

For example, the view list 42 is a list representing examination processes included in the examination protocol called an examination A. The view list 42 includes “View 1”, “View 2”, . . . , and “View m”. “View 1”, “View 2”, . . . , and “View m” are different examination processes and are arranged in accordance with the examination procedure. The examinations corresponding to the respective examination processes are executed in an order of “View 1”, “View 2”, . . . , and “View m”. For example, the B-mode image is captured or the measurement is performed in each of “View 1”, “View 2”, . . . , and “View m”. For example, an imaging condition or the like linked to the View is automatically set in the ultrasound diagnostic apparatus 10, and the ultrasonic examination corresponding to the View is executed. In this way, the imaging by the user is supported.

An image V1 is an image showing “View 1”. An image V2 is an image showing “View 2”. An image Vm is an image showing “View m”. For example, the images V1, V2, . . . , and Vm are images representing icons, character strings, and the like. The images V1, V2, . . . , and Vm are disposed and displayed in accordance with the examination procedure.

The controller 28 controls each unit of the ultrasound diagnostic apparatus 10 such that the ultrasonic examination is executed according to the examination protocol of the examination A. That is, the controller 28 controls each unit of the ultrasound diagnostic apparatus 10 such that the examination is executed in an order of “View 1”, “View 2”, . . . , and “View m”.

For example, the controller 28 performs various settings for executing the ultrasonic examination corresponding to “View 1”. In a case where “View 1” is an examination process of acquiring a B-mode image of a certain cross section, the controller 28 performs various settings for acquiring the B-mode image.

In a case where the ultrasound data (for example, the B-mode image) is acquired in the examination process called “View 1”, the linking unit 24 links the ultrasound data with information indicating “View 1” of the examination protocol of the examination A, and stores the ultrasound data and the information indicating “View 1” in the storage unit 22.

After “View 1” is completed, the controller 28 performs various settings for executing the ultrasonic examination corresponding to “View 2”. In a case where “View 2” is an examination process of acquiring a B-mode image of a certain cross section, the controller 28 performs various settings for acquiring the B-mode image.

In a case where the ultrasound data (for example, the B-mode image) is acquired in the examination process called “View 2”, the linking unit 24 links the ultrasound data with information indicating “View 2” of the examination protocol of the examination A, and stores the ultrasound data and the information indicating “View 2” in the storage unit 22.

Similarly, in a case where the ultrasound data is acquired in another examination process, the linking unit 24 links the acquired ultrasound data with information indicating the examination process (that is, the view) in which the ultrasound data is acquired, and stores the ultrasound data and the information in the storage unit 22. For example, in a case where the measurement is executed in the examination corresponding to a certain “View”, the linking unit 24 links the measurement result with information indicating the examination process in which the measurement result is acquired, and stores the measurement result and the information in the storage unit 22.

In this way, the examination corresponding to each examination process is executed in accordance with the order indicated by the view list 42, and the data obtained in the examination and the information indicating the examination process are linked with each other.

In the example shown in FIG. 2, ultrasound data “VIEWS2-1” and “VIEWS2-2” are acquired in “View 2”. An image V21 is an image showing the ultrasound data “VIEWS2-1”. An image V22 is an image showing “VIEWS2-2”. The images V21 and V22 are images representing icons, character strings, and the like. Since the ultrasound data “VIEWS2-1” and “VIEWS2-2” are acquired in “View 2”, the images V21 and V22 are displayed in a state of being linked to the image V2. For example, the images V21 and V22 are displayed near the image V2. Each of the ultrasound data “VIEWS2-1” and “VIEWS2-2” is stored in the storage unit 22 by being linked to information indicating “View 2”.

The ultrasound data acquired in each of “View 1”, “View 2”, . . . , and “View m” corresponds to an example of “first ultrasound data”.

In the example shown in FIG. 2, an additional examination “View?” is executed after the last examination process “View m”. An image Vx is an image showing the additional examination “View?”. The ultrasound data acquired in “View?” corresponds to an example of “second ultrasound data”.

For example, in a case where the user operates the operation unit 32 to issue an instruction to execute the additional examination, the controller 28 executes the additional examination in response to the instruction. In a case where the additional examination is an examination of acquiring the B-mode image, the B-mode image is acquired by executing the additional examination.

By moving the image Vx on the examination screen 34 by operating the operation unit 32, the user can link the second ultrasound data acquired in “View?” with the view (that is, the examination process) designated by the user. For example, as indicated by an arrow 44, the user can link the second ultrasound data with the view by moving the image Vx on the examination screen 34. The user may move the image Vx by operating the mouse, or may move the image Vx by a touch operation in a case where the examination screen 34 is configured with a touch panel.

For example, in a case where the image Vx is moved to a region between the image V2 showing “View 2” and an image showing “View 3” which is the next view, the linking unit 24 links the second ultrasound data acquired in “View?” with “View 2”. In this case, the linking unit 24 links the second ultrasound data with information indicating “View 2” and stores the second ultrasound data and the information in the storage unit 22.

Hereinafter, each example in which the second ultrasound data is linked to the examination process will be described.

### Example 1

In Example 1, the display controller 30 displays, on the display unit 20, an image schematically showing an examination site that is a candidate for the ultrasonic examination. The linking unit 24 links the second ultrasound data with an examination process corresponding to the examination site designated by the user from the image. Hereinafter, Example 1 will be described in detail.

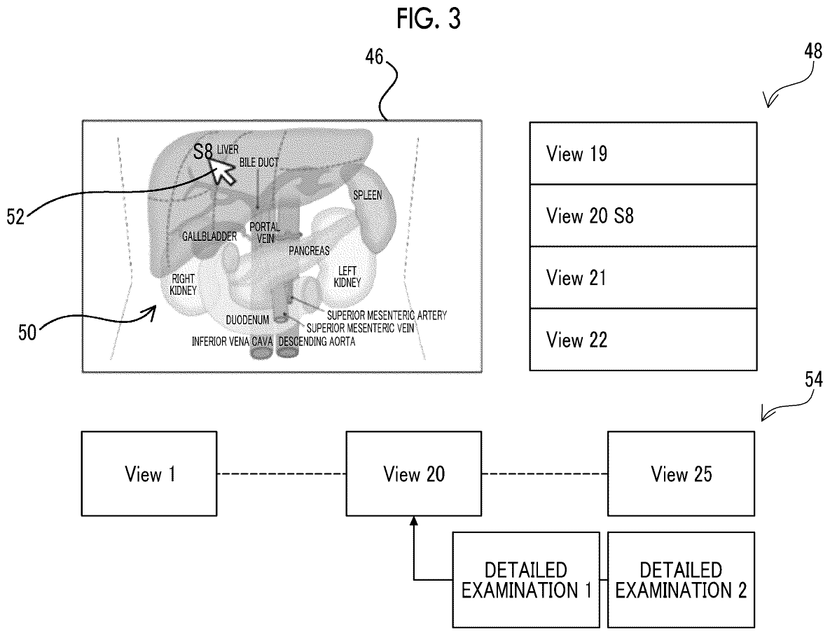

Example 1 will be described with reference to FIG. 3. FIG. 3 is a diagram for describing Example 1. FIG. 3 shows an examination screen 46 and a view list 48. The display controller 30 displays the examination screen 46 and the view list 48 on the display unit 20.

A pictogram 50 is displayed on the examination screen 46. The pictogram 50 is an example of an image schematically showing the examination site that is a candidate for the ultrasonic examination. The pictogram 50 is an image representing one or a plurality of organs. The organ represented by the pictogram 50 is an example of an examination site.

The organ represented by the pictogram 50 and a view (that is, an examination process) registered in the view list 48 are previously linked with each other. For example, for each organ, the organ and the view in which the organ is to be imaged are previously linked with each other. Information indicating the linkage is pre-registered in the examination protocol. By designating the organ represented by the pictogram 50, the view corresponding to the organ is designated.

In the example shown in FIG. 3, an organ S8 is designated by a pointer 52 by the user operating the operation unit 32. In a case where the examination screen 46 is configured with a touch panel, the organ S8 may be designated by a touch operation.

The organ S8 is previously linked to “View 20” registered in the view list 48. “View 20” is designated by designating the organ S8.

In the example shown in FIG. 3, “View 1” to “View 25” are pre-registered in the examination protocol. The view list 48 includes these “View”.

In a case where the user operates the operation unit 32 to issue an instruction to execute “Detailed Examination 1” and “Detailed Examination 2”, the ultrasound diagnostic apparatus 10 executes the ultrasonic examination corresponding to “Detailed Examination 1” and the ultrasonic examination corresponding to “Detailed Examination 2”. For example, a condition of the ultrasonic examination or the like is designated by the user. The ultrasound diagnostic apparatus 10 executes the ultrasonic examination corresponding to each “Detailed Examination” in accordance with the designated condition or the like. As a result, the second ultrasound data corresponding to “Detailed Examination 1” and the second ultrasound data corresponding to “Detailed Examination 2” are acquired.

In a case where the user designates the organ S8 from the pictogram 50 by operating the operation unit 32, the linking unit 24 links the second ultrasound data acquired in “Detailed Examination 1” and the second ultrasound data acquired in “Detailed Examination 2” with “View 20” linked to the organ S8. FIG. 3 schematically shows the state of the linkage as indicated by reference numeral 54. In a case where the user designates “View 20” included in the view list 48 by operating the operation unit 32, the linking unit 24 may link the second ultrasound data acquired in “Detailed Examination 1” and the second ultrasound data acquired in “Detailed Examination 2” with “View 20”. The linking unit 24 stores the information indicating the linkage in the storage unit 22.

In the example shown in FIG. 3, “View 20” that is already registered in the examination protocol is designated by the user, and the ultrasound data is linked to “View 20”. As another example, “View” may be added to the examination protocol, and the second ultrasound data may be linked to the added “View”.

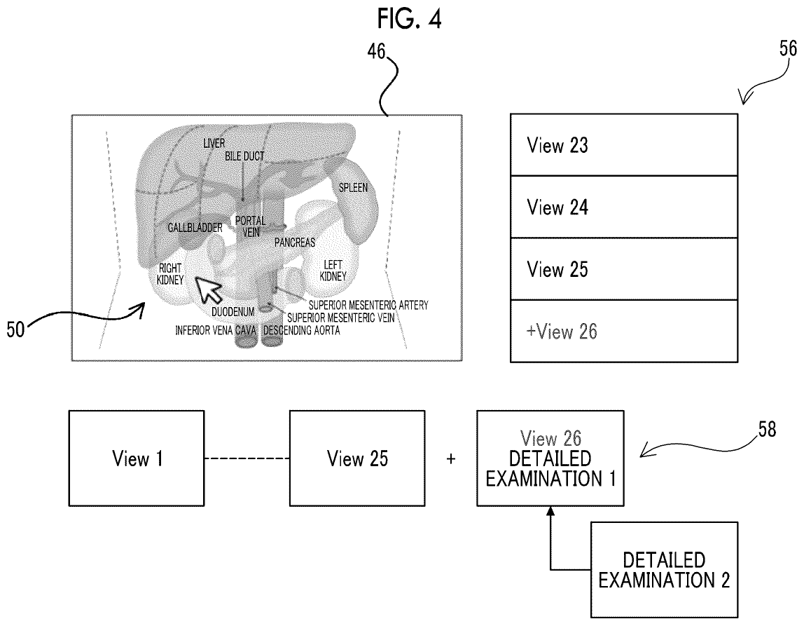

A process of linking the second ultrasound data with the “View” newly added will be described with reference to FIG. 4. FIG. 4 shows the examination screen 46 and a view list 56. The display controller 30 displays the examination screen 46 and the view list 56 on the display unit 20. In the example shown in FIG. 4, “View 1” to “View 25” are registered in the examination protocol. The view list 56 includes these “View”.

“Detailed Examination 1” and “Detailed Examination 2”, which are additional examinations, are executed in response to the instruction from the user, and the second ultrasound data is acquired in each of the ultrasonic examinations.

In a case where the user operates the operation unit 32 to issue an instruction to add “View”, the linking unit 24 creates new “View” and adds the new “View” to the examination protocol. In the example shown in FIG. 4, “View 26” is newly created and added to the examination protocol.

The linking unit 24 links the second ultrasound data acquired in “Detailed Examination 1” and the second ultrasound data acquired in “Detailed Examination 2” with “View 26”. FIG. 4 schematically shows the state of the linkage as indicated by reference numeral 58. The linking unit 24 stores the information indicating the linkage in the storage unit 22.

As described above, according to Example 1, the user can link the ultrasound data acquired by the additional ultrasonic examination with the ultrasonic examination by a simple operation such as an operation on the pictogram 50 or the view list.

### Example 2

In a case where the ultrasound data is acquired, accessory information is attached to the ultrasound data. For example, the controller 28 attaches the accessory information to the ultrasound data. The accessory information includes identification information (for example, a patient ID or information indicating a name) of the subject, information indicating an examination type, measurement information, setting information, and information indicating View (that is, the examination process), and the like.

For example, in a case where the ultrasonic examination is executed, the identification information of the subject is input to the ultrasound diagnostic apparatus 10. The information indicating the examination type, the setting information, and the information indicating View are previously linked to the ultrasonic examination. In a case where the ultrasonic examination is executed and the ultrasound data is acquired, the information is attached to the acquired ultrasound data as accessory information. In a case where the additional ultrasonic examination is executed to acquire the second ultrasound data, the accessory information is attached to the second ultrasound data.

In Example 2, in a case in which the examination process (that is, View) indicated by the accessory information attached to the second ultrasound data is different from the examination process designated by the user, the linking unit 24 links the examination process indicated by the accessory information with the second ultrasound data. In this manner, the linking unit 24 corrects the examination process linked to the second ultrasound data.

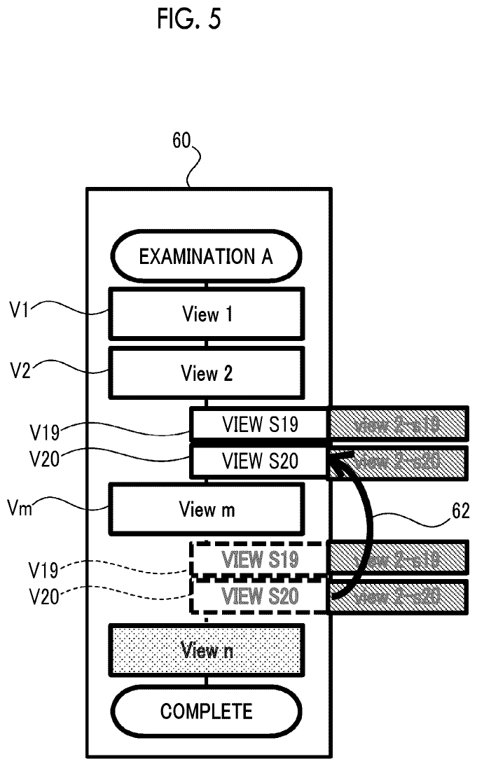

Example 2 will be described with reference to FIG. 5. FIG. 5 shows a view list 60. For example, ultrasound data “VIEWS19” and “VIEWS20” are acquired by additionally executing the ultrasonic examination corresponding to “View 2”. The ultrasound data “VIEWS19” and

“VIEWS20” are examples of the second ultrasound data. The accessory information is attached to the ultrasound data “VIEWS19” and “VIEWS20”. The accessory information includes information indicating “View 2”.

After the ultrasound data “VIEWS19” and “VIEWS20” are acquired, the ultrasound data “VIEWS19” and “VIEWS20” are linked to the examination process “View m” by the user. That is, information indicating “View m” is linked to each of the ultrasound data “VIEWS19” and “VIEWS20”.

“View m” is different from “View 2” indicated by the accessory information. In this case, the linking unit 24 releases “View m” linked to the ultrasound data “VIEWS19” and “VIEWS20” and links the ultrasound data “VIEWS19” and “VIEWS20” with “View 2”. In this manner, the linking unit 24 corrects the examination process linked to the ultrasound data “VIEWS19” and “VIEWS20”. As a result of this correction, “View 2” is linked to the ultrasound data “VIEWS19” and “VIEWS20”, and “View m” is not linked to the ultrasound data “VIEWS19” and “VIEWS20”.

For example, in a case where the user operates the operation unit 32 to issue an instruction to correct the examination process, the linking unit 24 corrects the view (that is, the examination process) linked to the ultrasound data “VIEWS19” and “VIEWS20” from “View m” to “View 2”.

In the example shown in FIG. 5, an image V19 is an image showing the ultrasound data “VIEWS19”. An image V20 is an image showing the ultrasound data “VIEWS20”. Since the ultrasound data “VIEWS19” and “VIEWS20” are linked to “View m” in response to the instruction from the user, the images V19 and V20 are linked to an image Vm showing “View m”. The linking unit 24 links the images V19 and V20 with the image V2 showing “View 2” by correcting the linkage. For example, as indicated by reference numeral 62, the images V19 and V20 are moved to the image V2 and are linked to the image V2.

As described above, according to Example 2, the ultrasound data is linked to the examination process based on the accessory information attached to the ultrasound data, and the ultrasound data is reorganized.

### Example 3

In Example 3, the linking unit 24 links the second ultrasound data and the examination process with each other based on the result of the analysis by the analysis unit 26.

The analysis unit 26 analyzes the second ultrasound data to specify the examination process corresponding to the ultrasonic examination in which the second ultrasound data is obtained. For example, the analysis unit 26 specifies a cross section or an examination site to be scanned with the ultrasonic waves in the ultrasonic examination. That is, the analysis unit 26 specifies a cross section or an examination site from which the second ultrasound data is acquired. In the ultrasonic examination, a cross section or an examination site to be scanned with the ultrasonic waves is predetermined, and the cross section or the examination site is previously linked to the ultrasonic examination. The ultrasonic examination in which the second ultrasound data is acquired is specified by specifying the cross section or the examination site from which the second ultrasound data is acquired.

For example, the analysis unit 26 applies a technique of analyzing a similar image to the second ultrasound data to specify the ultrasonic examination in which the second ultrasound data is obtained. As the technique of analyzing the similar image, artificial intelligence (AI) may be used, or a determination technique using statistical structure pattern recognition may be used. An image serving as a reference for the similar image may be reference data prepared previously, or may be data already acquired by executing the examination. A learning model may be used. The analysis unit 26 attaches the accessory information to the second ultrasound data based on the recognition result.

The linking unit 24 links the second ultrasound data with the examination process specified by the analysis unit 26.

For example, in a case where the second ultrasound data is acquired by the additional ultrasonic examination, the analysis unit 26 analyzes the second ultrasound data to specify the cross section or the examination site from which the second ultrasound data is acquired, and to specify the examination process corresponding to the ultrasonic examination in which the second ultrasound data is acquired. The linking unit 24 links the second ultrasound data with the specified examination process. For example, in a case where the examination process is “View 2”, the linking unit 24 links the second ultrasound data with the information indicating “View 2”.

For example, in a case where the user operates the operation unit 32 to issue an instruction to execute the processing according to Example 3, the processing according to Example 3 is executed, and the examination process is linked to the second ultrasound data.

According to Example 3, it is not necessary to perform work of manually linking the examination process with the second ultrasound data. Therefore, a burden on the user is reduced.

### Example 4

In Example 4, the display controller 30 arranges and displays an ultrasound data group before the examination process is corrected and an ultrasound data group after the examination process is corrected, on the display unit 20. The ultrasound data groups include the first ultrasound data and the second ultrasound data, that is, the display controller 30 arranges and displays the ultrasound data group before being organized and the ultrasound data group after being organized, on the display unit 20.

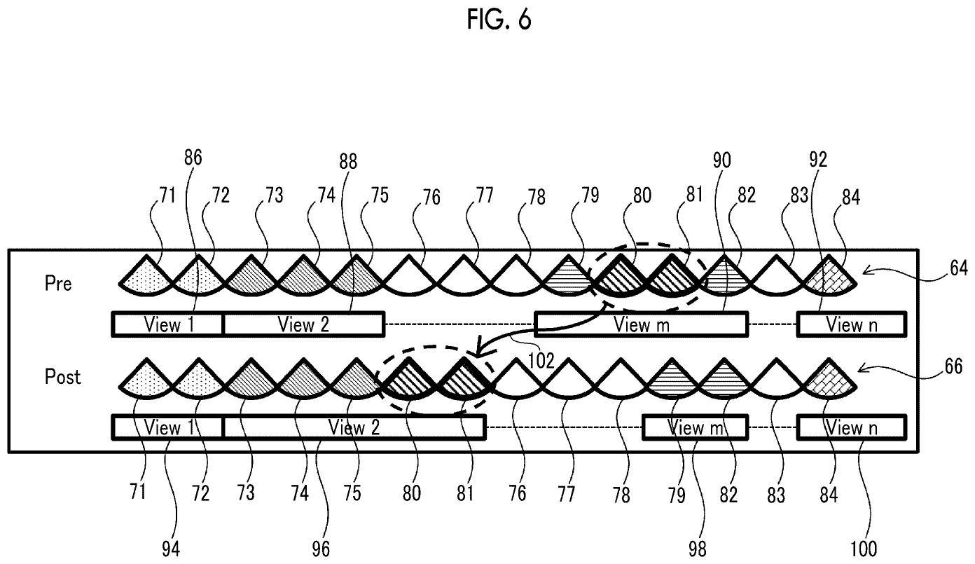

Example 4 will be described with reference to FIG. 6. FIG. 6 shows a display example of an ultrasound image. For example, the first ultrasound data and the second ultrasound data are a B-mode image or a color Doppler image. Of course, the first ultrasound data and the second ultrasound data are not limited to these images.

The display controller 30 generates a thumbnail image (that is, a minified image) of each of the first ultrasound data and the second ultrasound data. For example, the display controller 30 generates a thumbnail image of the ultrasound image such as the B-mode image or the color Doppler image.

The display controller 30 displays a plurality of thumbnail images before the examination process is corrected on the display unit 20 in accordance with an order of the examination processes, and displays a plurality of thumbnail images after the examination process is corrected on the display unit 20 in accordance with an order of the examination processes.

FIG. 6 shows a display example thereof. An image group 64 includes a plurality of thumbnail images before the linked examination process is corrected. That is, the image group 64 includes the plurality of thumbnail images before being organized. An image group 66 includes a plurality of thumbnail images after the linked examination process is corrected. That is, the image group 66 includes the plurality of thumbnail images after being organized. The display controller 30 arranges and displays the image group 64 and the image group 66 on the display unit 20.

The image groups 64 and 66 include thumbnail images 71 to 84. Each of the thumbnail images 71 to 84 is an image generated by reducing the ultrasound image such as the B-mode image or the color Doppler image. The thumbnail images 71 to 84 are thumbnail images generated from separate ultrasound data (for example, the ultrasound images such as the B-mode images or the color Doppler images).

Each of the thumbnail images 71 to 79 and 82 to 84 is a thumbnail image of the first ultrasound data. Each of the thumbnail images 80 and 81 is a thumbnail image of the second ultrasound data.

In addition, images 86 to 100 representing the examination process “View” are displayed. The images 86 to 92 are included in the image group 64. The images 94 to 100 are included in the image group 66.

The display controller 30 arranges and displays the images 86 to 92 representing “View” on the display unit 20 in accordance with an order of the examination processes registered in the examination protocol. The ultrasonic examination corresponding to each examination process is executed in an order of “View 1”, “View 2”, . . . , and “View m”, and “View n”. Therefore, the images 86 to 92 are displayed in that order.

Similarly, the display controller 30 arranges and displays the images 94 to 100 representing “View” on the display unit 20 in accordance with the order of the examination processes registered in the examination protocol. The ultrasonic examination corresponding to each examination process is executed in an order of “View 1”, “View 2”, . . . , and “View m”, and “View n”. Therefore, the images 94 to 100 are displayed in that order.

Each first ultrasound data and each second ultrasound data are linked to “View” (that is, the examination process). The display controller 30 links each thumbnail image with information indicating “View” in accordance with the linkage and stores the thumbnail image and the information on the display unit 20.

For example, the first ultrasound data serving as the original of the thumbnail image 71 and the first ultrasound data serving as the original of the thumbnail image 72 are ultrasound data acquired in the examination process called “View 1”. Therefore, the display controller 30 links the thumbnail images 71 and 72 with the image 86 representing “View 1” and displays the thumbnail images 71 and 72 and the image 86 on the display unit 20. For example, the display controller 30 displays the thumbnail images 71 and 72 near the image 86 representing “View 1”. The same applies to the thumbnail image corresponding to the other first ultrasound data.

The display controller 30 arranges and displays the thumbnail images 71 to 79 and 82 to 84 corresponding to the first ultrasound data on the display unit 20 in accordance with the order of the examination processes registered in the examination protocol.

The display controller 30 disposes the thumbnail images 80 and 81 corresponding to the second ultrasound data at positions corresponding to the order of the examination processes designated by the user in the image group 64, and displays the thumbnail images 80 and 81 on the display unit 20. The state of this disposition is a state before correction (that is, a state before organization). For example, since the examination process called “View m” is designated by the user, the display controller 30 displays the thumbnail images 80 and 81 near the image 90 representing “View m”.

As described in Example 2, after the examination process designated by the user is linked to the second ultrasound data, the examination process included in the accessory information is linked to the second ultrasound data, whereby the examination process linked to the second ultrasound data is corrected. That is, the examination processes are organized.

The second ultrasound data serving as the original of the thumbnail image 80 and the second ultrasound data serving as the original of the thumbnail image 81 are linked to the examination process called “View m” by the user, but the accessory information of these second ultrasound data includes information indicating the examination process called “View 2”. In this case, the linking unit 24 releases the linkage of “View m” from these second ultrasound data and links the examination process called “View 2” with these second ultrasound data. As a result, the linked examination process is corrected, and the examination processes are organized.

The display controller 30 disposes the thumbnail images 80 and 81 corresponding to the second ultrasound data at positions corresponding to the order of the examination processes after the correction in the image group 66, and displays the thumbnail images 80 and 81 on the display unit 20. The state of this disposition is a state after correction (that is, a state after organization).

As indicated by an arrow 102 in FIG. 6, the thumbnail images 80 and 81 are moved from “View m” before the correction to “View 2” after the correction, and are displayed near the image 96 representing “View 2”.

Since the image groups 64 and 66 are arranged and displayed, the user can visually check whether or not the correction (that is, the organization) of the linkage of the examination process is correctly performed.

For example, the thumbnail images 80 and 81 are displayed by being linked to “View m” before the correction, and are displayed by being linked to “View 2” after the correction. The user can visually recognize that the examination process linked to the second ultrasound data is changed from “View m” to “View 2” by referring to this display state.

As another example different from the above example, “View 2” linked to the thumbnail images 80 and 81 included in the image group 66 may be the examination process linked based on the analysis result by the analysis unit 26. That is, the display controller 30 may arrange and display the image group 64 including the thumbnail image of the second ultrasound data to which the examination process is linked by the user and the image group 66 including the thumbnail image of the second ultrasound data to which the examination process is linked based on the analysis result by the analysis unit 26 on the display unit 20. As a result, the user can compare and consider the result of the linkage by the user with the result of the linkage based on the analysis result.

### Example 5

In Example 5, the display controller 30 arranges and displays the first ultrasound data on the display unit 20 in accordance with an order of the examination processes linked to the first ultrasound data, and arranges and displays the second ultrasound data on the display unit 20 in accordance with an order of the examination processes linked to the second ultrasound data. The display controller 30 arranges and displays the first ultrasound data and the second ultrasound data on the display unit 20.

In a case where the user designates an examination process on the screen of the display unit 20 on which the first ultrasound data and the second ultrasound data are displayed, the linking unit 24 corrects the examination process linked to the second ultrasound data by linking the examination process designated by the user on the screen with the second ultrasound data. That is, the linking unit 24 releases the linkage of the examination process that was originally linked to the second ultrasound data and links the examination process designated by the user with the second ultrasound data. As a result, the examination process linked to the second ultrasound data is corrected. Information indicating the examination process that was originally linked to the second ultrasound data is not linked to the second ultrasound data, and information indicating the examination process designated by the user is linked to the second ultrasound data.

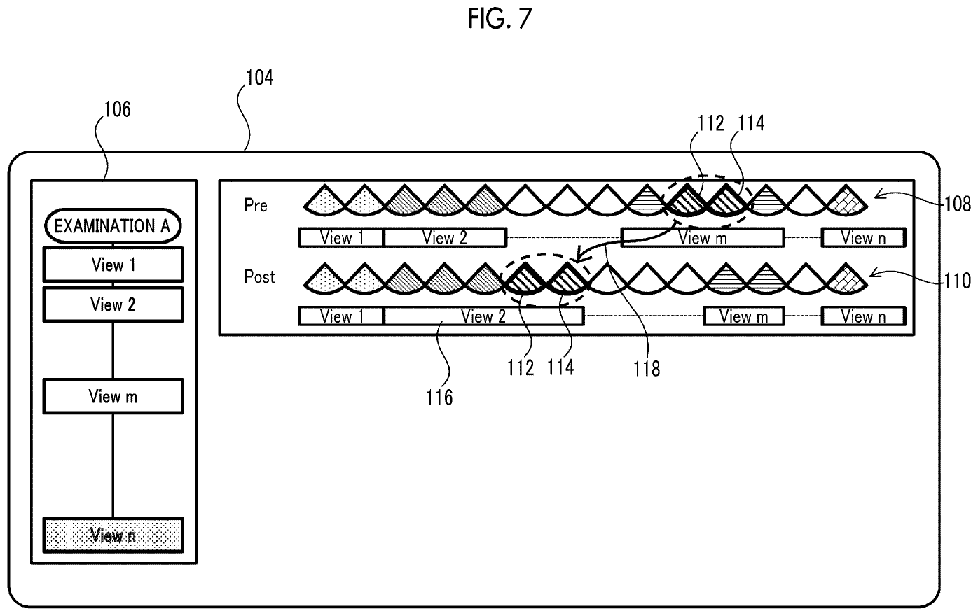

Example 5 will be described with reference to FIG. 7. FIG. 7 shows a display example of a view list and an ultrasound image.

A screen 104 is displayed on the display unit 20. For example, in a case where the user issues an instruction to display the screen 104, the display controller 30 displays the screen 104 on the display unit 20. A view list 106, an image group 108, and an image group 110 are displayed on the screen 104.

The view list 106 includes a plurality of Views registered in the examination protocol of the examination A.

Each of the image groups 108 and 110 includes a plurality of thumbnail images, as with the image groups 64 and 66 (see FIG. 6). Each thumbnail image is a minified image of ultrasound data (for example, an ultrasound image such as a B-mode image or a color Doppler image). The image group 108 is an image group before correcting the linkage of the examination process. The image group 110 is an image group after the linkage of the examination process is corrected.

Among the plurality of thumbnail images included in the image groups 108 and 110, thumbnail images 112 and 114 are thumbnail images of the second ultrasound data. Thumbnail images other than the thumbnail images 112 and 114 are thumbnail images of the first ultrasound data.

As in the display example of FIG. 6, the respective thumbnail images corresponding to the first ultrasound data are arranged and displayed in accordance with the order of the examination processes registered in the examination protocol.

In addition, as in the display example of FIG. 6, an image representing “View” is displayed. For example, an image representing “View 1”, an image representing “View 2”, . . . , and an image representing “View m”, and an image representing “View n” are included in each of the image groups 108 and 110, and are arranged and displayed in the order of the examination processes.

Before correcting the linkage of the examination process, the thumbnail images 112 and 114 are displayed by being linked to the image representing “View m” (see the image group 108). That is, the second ultrasound data corresponding to each of the thumbnail images 112 and 114 is linked to the examination process called “View m”. This linkage may be performed by the user or may be automatically performed by the analysis unit 26 or the like.

The user can correct the examination process linked to the ultrasound data according to the following procedure.

First, the user operates the operation unit 32 to select a thumbnail image to be corrected from the image group 108. The user may select the thumbnail image by operating a device such as a mouse, or may select the thumbnail image by a touch operation on a touch panel. Here, as an example, the thumbnail images 112 and 114 corresponding to the second ultrasound data are selected.

Next, the user operates the operation unit 32 to select the image representing “View” corresponding to the examination process after the correction, from the image group representing the “View” included in the image group 110. For example, in a case where the user selects an image 116 representing “View 2”, the display controller 30 moves the thumbnail images 112 and 114 from a display position of the images representing “View m” in the image group 108 to a display position of the image 116 representing “View 2” in the image group 110, as indicated by an arrow 118. As a result, the thumbnail images 112 and 114 are displayed near the image representing “View 2” in the image group 110. The linking unit 24 links the second ultrasound data corresponding to each of the thumbnail images 112 and 114 with the examination process called “View 2”. That is, the linking unit 24 deletes the information indicating “View m” linked to the second ultrasound data corresponding to each of the thumbnail images 112 and 114, and links the second ultrasound data corresponding to each of the thumbnail images 112 and 114 with the information indicating “View 2”. In this way, the examination process linked to the second ultrasound data is corrected.

The user may correct the examination process linked to the ultrasound data by moving the thumbnail image on the screen 104. For example, the user operates the operation unit 32 to select the thumbnail images 112 and 114 included in the image group 108. The user drags the selected thumbnail images 112 and 114 to a position of the image 116 representing “View 2” and drops the selected thumbnail images at the position. The linking unit 24 corrects the examination process linked to the second ultrasound data corresponding to each of the thumbnail images 112 and 114 to the examination process called “View 2”. In a case where the screen 104 is configured with a touch panel, the user may move the thumbnail images 112 and 114 by a touch operation, a slide operation, or the like.

The examination process linked to the first ultrasound data may be corrected. For example, the user moves the thumbnail image of the first ultrasound data from a display position of the image showing “View” before the correction to a display position of the image showing “View” after the correction. In this case, the linking unit 24 releases the linkage between the first ultrasound data and “View” before the correction, and links the first ultrasound data with “View” after the correction. Of course, the examination process linked to the first ultrasound data may be corrected by designating an image showing “View” after the correction by the user.

### Example 6

As another example different from Example 5, “View 2” linked to the thumbnail images 112 and 114 included in the image group 110 may be the examination process linked based on the analysis result by the analysis unit 26. “View 2” linked to the thumbnail images 112 and 114 included in the image group 108 is the examination process linked by the user.

That is, the image group 108 includes the thumbnail images 112 and 114 of the second ultrasound data to which the examination process is linked by the user. The image group 110 includes the thumbnail images 112 and 114 of the second ultrasound data to which the examination process is linked based on the analysis result by the analysis unit 26.

Similarly to Example 5, in a case where the user selects the thumbnail images 112 and 114 from the image group 108 and selects the examination process, the linking unit 24 links the second ultrasound data corresponding to each of the thumbnail images 112 and 114 with the the examination process. As a result, examination process linked to the second selected ultrasound data is corrected. Similar to Example 5, the examination process after the correction may be selected by moving the thumbnail image by a drag-and-drop operation, a slide operation, or the like on the thumbnail image.

According to Example 6, the user can compare the examination process linked to the second ultrasound data by the user with the examination process automatically linked to the second ultrasound data based on the analysis result of the analysis unit 26, by comparing the image groups 108 and 110.

The signal processing unit 16, the display processing unit 18, the linking unit 24, the analysis unit 26, the controller 28, and the display controller 30 can be realized by using, for example, hardware resources such as a processor and an electronic circuit, and a device such as a memory may be used in the realization as necessary. In addition, the signal processing unit 16, the display processing unit 18, the linking unit 24, the analysis unit 26, the controller 28, and the display controller 30 may be realized by a computer, for example. That is, all or a part of the signal processing unit 16, the display processing unit 18, the linking unit 24, the analysis unit 26, the controller 28, and the display controller 30 may be realized by cooperation between hardware resources, such as a central processing unit (CPU) or a memory included in a computer, and software (program) that defines the operation of the CPU or the like. The program is stored in the storage unit 22 of the ultrasound diagnostic apparatus 10 or other storage device through a recording medium, such as a CD or a DVD, or a communication path, such as a network. As another example, the signal processing unit 16, the display processing unit 18, the linking unit 24, the analysis unit 26, the controller 28, and the display controller 30 may be realized by a digital signal processor (DSP), an application specific integrated circuit (ASIC), a field programmable gate array (FPGA), or the like. Of course, a graphics processing unit (GPU) and the like may be used. The signal processing unit 16, the display processing unit 18, the linking unit 24, the analysis unit 26, the controller 28, and the display controller 30 may be realized by a single apparatus, or each of functions of the signal processing unit 16, the display processing unit 18, the linking unit 24, the analysis unit 26, the controller 28, and the display controller 30 may be realized by one or a plurality of apparatuses.

The linkage by the linking unit 24 or the analysis by the analysis unit 26 may be executed by an apparatus (for example, a personal computer or a server) other than the ultrasound diagnostic apparatus 10.

What is claimed is:

1. An ultrasound diagnostic apparatus comprising:
an acquisition unit that acquires ultrasound data by executing an ultrasonic examination; and
a linking unit that links first ultrasound data, which is acquired by the acquisition unit by executing the ultrasonic examination in accordance with a pre-registered examination procedure, with an examination process corresponding to the ultrasonic examination,
wherein the linking unit further links second ultrasound data, which is acquired by executing the ultrasonic examination in a procedure different from the pre-registered examination procedure, with an examination process corresponding to the ultrasonic examination.

2. The ultrasound diagnostic apparatus according to claim 1,
wherein the linking unit links an examination process designated by a user with the second ultrasound data.

3. The ultrasound diagnostic apparatus according to claim 2, further comprising:
a display controller that displays, on a display unit, an image schematically showing an examination site that is a candidate for the ultrasonic examination,
wherein the linking unit links an examination process corresponding to the examination site designated by the user from the image with the second ultrasound data.

4. The ultrasound diagnostic apparatus according to claim 2, further comprising:
a display controller,
wherein the display controller arranges and displays the first ultrasound data on a display unit in accordance with an order of the examination processes linked to the first ultrasound data, and arranges and displays the second ultrasound data on the display unit in accordance with an order of the examination processes linked to the second ultrasound data, and
in a case where the user designates an examination process on a screen on which the first ultrasound data and the second ultrasound data are displayed, the linking unit corrects the examination process linked to the second ultrasound data by linking the examination process designated by the user on the screen with the second ultrasound data.

5. The ultrasound diagnostic apparatus according to claim 2,
wherein, in a case where the second ultrasound data is acquired by the acquisition unit, information indicating the examination process in which the second ultrasound data is acquired is attached to the second ultrasound data as accessory information, and
in a case where the examination process indicated by the accessory information is different from the examination process designated by the user, the linking unit corrects the examination process linked to the second ultrasound data by linking the examination process indicated by the accessory information with the second ultrasound data.

6. The ultrasound diagnostic apparatus according to claim 5, further comprising:
a display controller,
wherein the display controller arranges and displays, on a display unit, an ultrasound data group before the examination process is corrected and an ultrasound data group after the examination process is corrected, and
the ultrasound data groups include the first ultrasound data and the second ultrasound data.

7. The ultrasound diagnostic apparatus according to claim 1, further comprising:
an analysis unit that analyzes the second ultrasound data to specify the examination process corresponding to the ultrasonic examination in which the second ultrasound data is obtained,
wherein the linking unit links the examination process specified by the analysis unit with the second ultrasound data.

8. The ultrasound diagnostic apparatus according to claim 7, further comprising:
a display controller,
wherein the display controller displays the first ultrasound data on a display unit in accordance with an order of examination processes, displays the second ultrasound data on the display unit in accordance with an order of examination processes designated by a user, and displays the second ultrasound data on the display unit in accordance with an order of the examination processes which are specified by the analysis unit and correspond to the ultrasonic examination, and
in a case where the user designates an examination process on a screen on which the first ultrasound data and the second ultrasound data are displayed, the linking unit corrects the examination process linked to the second ultrasound data by linking the examination process designated by the user on the screen with the second ultrasound data.
